(19) United States
(12) Patent Application Publication
(10) Pub. No.: US 2026/0020852 A1
(43) Pub. Date: Jan. 22, 2026

(54) DEVICE FOR REDUCING THE RETRACTION OF THE EDGES AND/OR FOR STRETCHING THE EDGES OF AN OPENED TISSUE STRUCTURE

(71) Applicant: ZACZEK MEDIZINTECHNIK UG, Bochum (DE)

(72) Inventor: Michael ZACZEK, Bochum (DE)

(21) Appl. No.: 18/997,451

(22) PCT Filed: Aug. 11, 2023
(86) PCT No.: PCT/EP2023/072349

§ 371 Date: Jan. 21, 2025

(30) Foreign Application Priority Data
Aug. 19, 2022 (DE) 10 2022 121 046.4

(51) Int. Cl. A61B 17/04; A61B 17/06; A61F 13/05

(52) U.S. Cl. CPC A61B 17/0487; A61B 17/06166; A61F 13/05; A61B 2017/0464; A61B 2017/0496

(57) ABSTRACT

The present invention relates to a device for reducing the retraction of the edges and/or for stretching the edges of an opened tissue structure of a patient with an open soft tissue defect, whereby the device has at least a base body, at least one retraction element for retracting and attaching to the edges of the opened tissue structure and a clamping element, wherein the clamping element is designed and arranged in such a way that the at least one retraction element can be moved by means of the clamping element in such a way that the edges of the opened tissue structure are at least partially brought together. The present invention further relates to a wound care system, comprising at least one such device and a vacuum dressing system, wherein the vacuum dressing system comprises at least one foam sheet dressing and a vacuum pump.

### CROSS REFERENCE TO RELATED APPLICATION

The present application is a U.S. National Phase of International Application No. PCT/EP2023/072349 entitled “DEVICE FOR REDUCING THE RETRACTION OF THE EDGES AND/OR FOR STRETCHING THE EDGES OF AN OPENED TISSUE STRUCTURE,” and filed on Aug. 11, 2023. International Application No. PCT/EP2023/072349 claims priority to German Patent Application No. 10 2022 121 046.4 filed on Aug. 19, 2022. The entire contents of each of the above-listed applications are hereby incorporated by reference for all purposes.

### TECHNICAL FIELD

The present invention relates to a device for reducing retraction of the edges and/or a device for stretching the edges of an opened tissue structure of a patient with an open soft tissue defect.

### BACKGROUND AND SUMMARY

The present invention also relates to a wound care system comprising a device for reducing the retraction of the edges and/or a device for stretching the edges of an opened tissue structure of a patient with an open soft tissue defect and a vacuum dressing system.

After visceral surgery, it may be that after completion of the actual procedure, closure of the abdominal wall is not possible or is not even desired. This may be the case, for example, if the patient has a severely contaminated abdominal cavity, as may be the case, for example, with perforation of a hollow organ. In such cases, a repeat lavage of the abdominal cavity (especially peritoneal lavage) may be indicated.

It is also conceivable that in the case of hollow organ injuries that cannot be treated surgically, such as small bowel fistulas, the abdominal wall cannot or should not be closed initially.

Other indications include, for example, ileus in which the intestines require so much space despite decompression that the fascia in the abdominal wall area cannot be closed.

It is also conceivable that in the event of a risk of abdominal compartment syndrome, the abdominal cavity is not closed again initially. Abdominal compartment syndrome is a life-threatening condition that occurs as a result of a rapidly progressive increase in intra-abdominal pressure (in particular IAP>12 mm Hg) and results in serious impairment of intra-and extra-abdominal organ systems.

Further indications are conceivable, particularly for the treatment of large hernias.

Retraction devices for treating open abdomens are already known from the prior art, by means of which the wound edges of an opened abdominal cavity can be retracted.

An example of this is the wound care device according to WO2022074032A1, which comprises a device for reducing retraction of the edges of an opened fascia after/during an open abdomen.

However, such devices are unfavorable in that they only reduce the retraction of the edges of an opened tissue structure of a patient, whereas an actual approach of the edges would be more favorable for a re-closure of the tissue structure.

Furthermore, these devices require a great deal of space, in particular a great deal of vertical space (from the perspective of a patient lying down), which is especially problematic if the patient requires intensive care. Also, repositioning of the patient is significantly restricted (up to 30° lateral tilt), which on the one hand increases the risk of pressure ulcers, and on the other hand makes normal care routines and ensuring the patient's personal hygiene more difficult.

It is therefore the object of the present invention to advantageously further develop a device for reducing the retraction of the edges and/or for stretching the opened tissue structure of a patient of the type mentioned at the beginning, in particular to the effect that a better closure of an opened tissue structure of a patient can be achieved.

This problem is solved by the invention by means of a device with the features of claim 1. Accordingly, a device is provided for reducing the retraction of the edges and/or for stretching an opened tissue structure of a patient with an open soft tissue defect, whereby the device comprises at least one base body, at least one retraction element for retracting and attaching to the edges of the opened tissue structure and a clamping element, wherein the clamping element is designed and arranged in such a way that the at least one retraction element can be moved by means of the clamping element, in particular continuously and/or with a predefined constant force, in such a way that the edges of the opened tissue structure are at least partially brought together.

The invention is based on the basic idea of being able to bring the edges of an opened tissue structure closer together or to pull them together. This process of pulling together should not be abrupt and should not be done with too much force. Rather, it becomes possible to at least partially bring the edges of the wound together by pulling them relatively slowly, possibly with predefined and/or appropriate force. Accordingly, not only can the retraction of the edges of the opened tissue structure be reduced, but closure of the opened tissue structure can be facilitated or improved by the said approach of the edges.

An edge of an opened tissue structure refers to both the outermost boundary of a tissue (skin, muscles, fasciae, tendon plates, fat, etc.) located in front of an opened tissue structure and a few centimeters lateral (away from the center of the body) of it.

The device can be designed and set up in such a way that the at least one retraction element can be moved by means of the clamping element essentially in a plane with the opened tissue structure or essentially parallel to the opened tissue structure. In other words, the pulling movement can be carried out more or less at the level or in the plane of the edges of the opened tissue structure. Consequently, the device requires little space, particularly vertically (from the perspective of a patient lying down), which is particularly advantageous if a patient requires intensive care. It is also possible to reposition the patient, which on the one hand reduces the risk of pressure ulcers and on the other hand also enables normal care routines and ensures the patient's personal hygiene.

The clamping element can be designed and set up in such a way that the at least one retraction element is moved with a force of up to 10 N by means of the clamping element. A force of this magnitude not only makes it possible to reduce the edges of the opened tissue structure, but also makes it possible to at least partially bring the edges of the opened tissue structure closer together.

In particular, it may be further provided that the device comprises at least two retraction elements. Alternatively, it is conceivable that the device comprises a retraction element with several (at least two) sections. Several retraction elements or several sections of a retraction element can enable better approach of the edges of the opened tissue structure.

Furthermore, it can be provided that the base body is arranged essentially centrally between the at least two retraction elements. Thus, opposite edges of an opened tissue structure can be brought closer together.

In particular, it is conceivable that the base body is directly and/or indirectly connected at at least one point to the at least one retraction element or to the at least two retraction elements. This makes it possible for a force to be transmitted from the clamping element via the base body to the at least one or the at least two retraction elements, and from there to the edges of the opened tissue structure. Alternatively and/or additionally, it is conceivable that the base body guides and/or deflects the at least one retraction element.

The base body can have a substantially elongated shape. Tissue openings are often substantially elongated due to a surgical incision. A base body with a substantially elongated shape is therefore largely compatible with an elongated tissue opening. This allows the edges of the tissue opening to be brought together along an axis, so that only one suture (longitudinal) is required to close the tissue opening.

In particular, it is conceivable that the base body is essentially in the form of a cuboid, a cylinder or a spindle. A base body in the form of a cylinder or a spindle has the advantage that a retraction element in the form of a thread can be easily wrapped around the cylinder or the spindle.

Furthermore, the base body can comprise a winding mechanism. This makes it possible to pull the edges of the opened tissue structure towards the center of the tissue opening. Alternatively, the clamping element can comprise a winding mechanism.

It is also conceivable that the base body comprises at least two partial bodies that can be releasably connected to one another. The two partial bodies can be releasably connected to one another, for example, by screws, clips, clicks, plugs, ties and/or magnetism.

In particular, it is conceivable that the base body comprises two partial bodies which are detachably connected to each other, for example along their longitudinal axis.

Each partial body can be connected to at least one retraction element and/or designed in such a way that it is set up to deflect at least one retraction element.

In other words, each partial body can comprise at least one deflection element that is set up to deflect the at least one retraction element. Alternatively, it is possible that one or more deflection units are attached to a partial body.

It is conceivable that at least one retraction element can be guided through the partial body and deflected therein via the deflection element. Each partial body can thus be connected to an edge of an opened tissue structure by at least one retraction element. By loosening the connection of the two partial bodies, on the one hand the tension on the edges of the opened tissue structure can be reduced, and on the other hand access to the opened tissue structure (for example for wound cleaning processes and/or for examinations) and/or to a vacuum dressing can be made possible.

In particular, it may be envisaged that the base body is made of autoclavable or sterilizable material or comprises an autoclavable or sterilizable material and remains on the patient throughout the entire therapy.

Furthermore, it is conceivable that the clamping element comprises a spring and/or a motor. A spring offers the advantage of a mechanical drive and can be designed in such a way that the clamping element and the device can be autoclaved. A motor can enable electrical control of the clamping element. Furthermore, a hydraulic and/or pneumatic drive of the clamping element would also be conceivable.

The clamping element can be at least partially attached in or on the base body. This enables a space-saving design of the device, in particular of the combination of clamping element and base body. Attaching the clamping element in the base body also makes it easy to clean the device, since only the base body needs to be cleaned.

Alternatively, it is possible that the clamping element is not attached to the base body, but is connected or can be connected to the base body via at least one retraction element.

It is possible that the clamping element comprises a chamber, in particular a hydraulic and/or pneumatic chamber. The chamber can be connected to a pump directly or indirectly.

The presence of a pneumatic chamber allows a patient's coughing jolts to be cushioned by briefly immersing the cylinders.

At least one pressure element can be connected to the chamber. There can also be at least one pressure element in the chamber. The pressure element can be in the form of a cuboid or cylinder, for example.

The pressure element can be connected to at least one retraction element, which is directly or indirectly connected to an edge of an opened tissue structure. Via a pump connected to the chamber, the at least one pressure element can be moved in such a way that a tensile force is exerted on the edge of the opened tissue structure via the at least one retraction element connected to the pressure element.

If the device comprises a clamping element with several pressure elements, which are each connected to at least one retraction element, each retraction element can experience the same tensile force, possibly also continuously. By connecting the chamber to a pump (for example, an electric pump or a manual pump), the pressure in the chamber and thus the tensile force of the at least one retraction element can be varied as required from outside. The connection can be, for example, a pneumatic or hydraulic connection, e.g. one or more tubes. This can enable flexible adaptation to different wound healing stages, tissue properties, patient needs, etc.

The chamber can be arranged outside of the soft tissue defect or the opened tissue structure and/or away from a dressing located in the opened tissue structure. It is conceivable that the chamber is attached under a sterile film near the opened tissue structure.

The at least one retraction element can comprise at least one thread and/or suture material and/or at least one mesh and/or at least one mesh strip and/or at least one clip and/or at least one hook and/or at least one tube, for example a Bowden-PTFE tube, and/or at least one wire. Several of the mentioned elements can also be combined to form a retraction element. By selecting one of the mentioned retraction elements or combining at least two of the mentioned retraction elements, an individual adaptation of the device to the nature of the tissue opening to be treated is achieved.

The device can comprise several retraction elements of different lengths and/or sizes. In particular, it is conceivable that, when the base body is located in the center of the tissue opening, the distance between the base body and the edges of the tissue opening is different. Retraction elements of different sizes and/or lengths can be used to enable even traction on the edges. In particular, the length of the retraction elements can be adapted to a varying wound dehiscence. Wound dehiscence is the undesired divergence of the wound edges. The extent and depth of the wound gaping depends on the severity of the wound dehiscence.

In particular, it is conceivable that a retraction element comprises at least one surgical thread and at least one mesh or mesh strip and/or at least one hook. The at least one mesh can be connected on one side to an edge of the opened tissue structure by numerous attachment points (for example by hooks, sutures and/or clips) and on the other side to a thread (thread pulled through the meshes of the mesh or fixed with a hook or clip), which in turn is connected to the base body. This enables a very even distribution of force through the numerous attachment points on the edge of the opened tissue structure (fascia). Furthermore, there may be significantly less resistance when the thread slides through the mesh. The total tensile force exerted by the device can also be controlled by varying the number of stitches on the mesh (i.e. the points where the thread connects to the mesh). The more stitches there are (where the thread is attached to the mesh), the greater the force. Furthermore, the mesh interposition (between the tissue and the thread) can be used as an additional seam bearing for the permanent closure of the opened tissue structure at the end of the device's use on the patient.

The invention further comprises a wound care system comprising at least one device according to the invention, wherein the wound care system further comprises a vacuum dressing system, wherein the vacuum dressing system comprises at least one foam film dressing and a vacuum pump. Vacuum dressing systems can be provided by different manufacturers and/or suppliers or can be individually manufactured. Combining the device according to the invention with the vacuum dressing system can enable improved wound healing. The use of vacuum dressings in the open abdomen is associated with a reduced incidence of enteroatmospheric fistulas, which is why their use should be aimed for.

The vacuum dressing system can also be referred to as a vacuum-assisted wound conditioning and cleaning system. The principle of vacuum-assisted treatment is very simple: a foam (or other wound filler, e.g. a sponge) is cut to approximately the size of the tissue opening and placed in the tissue opening. The wound area is covered with at least one film and connected to a vacuum by means of a drainage tube that is attached to the foam or similar.

The combination of foam or similar and film can be referred to as a foam-film dressing. The foam ensures that the vacuum applied is distributed evenly over the entire surface of the tissue opening. The suction provokes active wound cleansing by removing excess wound exudate, cell debris and bacteria from the wound. Wound edema is reduced and thus the blood flow situation in the wound area is improved. In addition, the formation of granulation tissue is accelerated.

In particular, it is conceivable that the device according to the invention is at least partially integrated into the foam film dressing or at least partially applied to it. The device can develop its full effect. This has the advantage that the patient treated with the wound care system is restricted by it as little as possible. This in turn enables improved positioning conditions, as well as improved conditions for the care of the patient.

Further details and advantages of the inventions will now be explained on the basis of the examples shown in the drawings.

### BRIEF DESCRIPTION OF THE FIGURES

It is shown in:

FIG. 1 an exemplary representation of an embodiment of an apparatus according to the invention for reducing the retraction of the edges and/or stretching the edges of an opened tissue structure of a patient with an open soft tissue defect;

FIG. 2 is another exemplary representation of an apparatus for reducing the retraction of the edges and/or stretching the edges of an opened tissue structure of a patient with an open soft tissue defect;

FIG. 3 an exemplary representation of a wound care system according to the invention;

FIG. 4 an exemplary representation of a further embodiment of an apparatus for reducing the retraction of the edges and/or stretching the edges of an opened tissue structure of a patient with an open soft tissue defect (on an abdominal wall simulator);

FIG. 5 a representation of an embodiment of a clamping element 106 with a chamber 118 and five pressure elements 120 in the form of cylinders 120;

FIG. 6 schematically a further embodiment of a clamping element 106;

FIG. 7 exemplary various retraction elements 104;

FIG. 8 schematically a further embodiment of a clamping element 106; and

FIG. 9 exemplary a detailed view of a pressure element 120.

### DETAILED DESCRIPTION

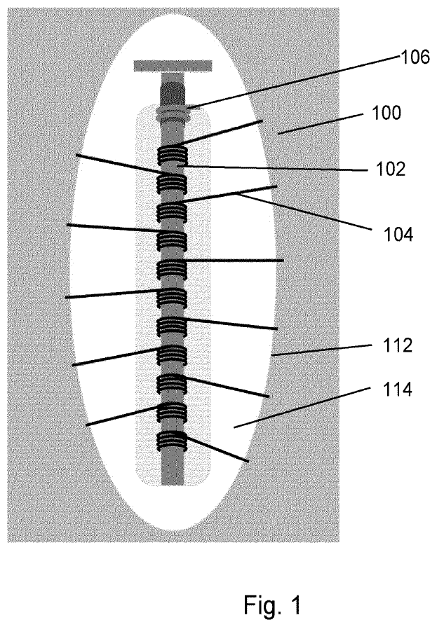

FIG. 1 illustrates an embodiment of an apparatus 100 according to the invention in an opened tissue structure 114, namely between the edges 112 of the opened tissue structure.

The opened tissue structure 114 is an open abdomen.

The device 100 comprises a base body 102.

In this embodiment, the base body 102 has an elongated shape.

In this embodiment, the base body 102 has the shape of a cylinder, but it could alternatively have the shape of a spindle, for example.

The device 100 also includes several retraction elements 104, which are designed for retraction and attachment to the edges 112 of the opened tissue structure 114.

Alternatively, the device 100 could comprise only one retraction element 104 or a retraction element 104 with several sections.

The base body 102 is arranged here centrally, i.e. essentially in the middle, between the retraction elements 104.

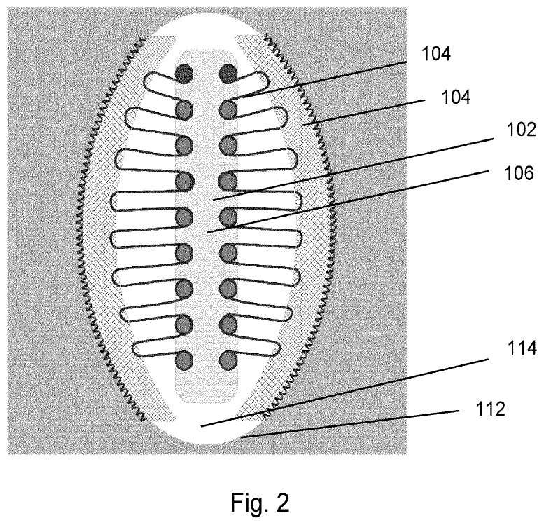

In this embodiment, the retraction elements 104 are threads, more specifically surgical threads. Alternatively, another suture material or at least one mesh and/or at least one mesh strip and/or at least one clamp or at least one hook or at least one tube or at least one wire would be conceivable. However, a combination of different retraction elements 104 would also be conceivable, see FIG. 2.

In this embodiment, the threads 104 are of equal length, but alternatively the threads 104 could also have different lengths.

In addition, the device includes a clamping element 106.

The clamping element 106 here includes a spring 106. In addition or alternatively, the clamping element 106 could also include a motor.

Here, the spring 106 is attached to the base body 102.

This connects the spring 106 to the base body 102. In particular, the spring 106 can thus transfer force to the base body 102.

Alternatively, the spring 106 could be at least partially mounted in the base body 102.

The base body 102 is directly connected at one point to one end of a thread 104 (here by knotting). Alternatively, an indirect connection, e.g. via an intermediate element, would be conceivable.

The threads 104 are each connected to an edge 112 of the opened tissue structure 114 by the end that is not connected to the base body 102.

In this embodiment, the threads 104 are knotted to/with the edges 112 of the opened tissue structure 114.

The spring 106 moves the base body 102 along its longitudinal axis (by tensioning force).

The base body 102 is thereby rotated along its longitudinal axis.

As a result, the spring 106 moves the threads 104 continuously and with a predefined constant force.

In this embodiment, the spring 106 moves the threads 104 with a force of 10 N.

Alternatively, the spring 106 can also move the threads 104 with a force of less than 10 N.

In this embodiment, the threads 104 are wound onto the cylinder 102.

This causes the edges 112 of the opened tissue structure 114 to be brought closer together, at least partially.

Retraction of the edges 112 is thus reduced.

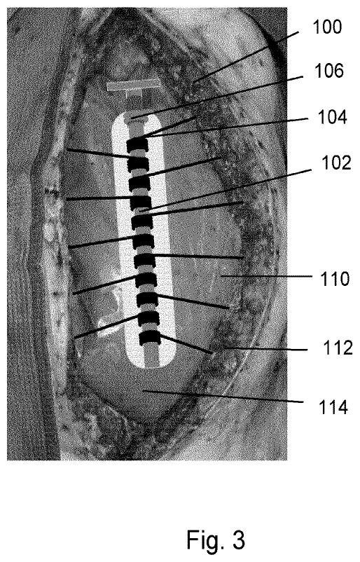

In this embodiment, the spring 106 moves the threads 104 in a plane with the opened tissue structure 114. Alternatively, the spring 106 can move the threads 104 parallel to the tissue structure 114 (as is the case in FIG. 3).

FIG. 2 shows another exemplary representation of an embodiment of an apparatus for reducing the retraction of the edges and/or stretching the edges of an opened tissue structure of a patient having an open soft tissue defect.

Shown here is also an apparatus 100 according to the invention in an opened tissue structure 114, namely between the edges 112 of the opened tissue structure 114.

The apparatus 100 comprises a base body 102 and a clamping element 106 (clamping element 106 not explicitly shown).

In this embodiment, the clamping element 106 is a winch that is located in the base body 102.

The base body 102 thus includes a winch.

In this embodiment, pulleys are attached to the base body 102.

The device 100 further comprises a plurality of retraction elements 104 adapted for retraction and attachment to the edges 112 of the opened tissue structure 114.

Here, there is a combination of different retraction elements 104, namely a combination of surgical threads 104 and meshes 104. In particular, there is a combination of two surgical threads 104 and two meshes 104.

The base body 102 is arranged centrally here, i.e. essentially in the middle, between the retraction elements 104.

In each case, one mesh 104 is connected to one edge 112 of the opened tissue structure 114. In this embodiment, one mesh 104 is knotted on one side to/with one edge 112 of the opened tissue structure 114 by surgical threads. The other side of each one of the meshes 104 is connected to a surgical thread 104.

A surgical thread 104 is threaded through the meshes of each one of the meshes 104 (on the side facing away from the tissue edge 112).

In addition, a surgical thread 104 is connected to the base body 102 via the deflection rollers. In other words: a surgical thread 104 is wound around deflection rollers on the base body 102.

The winch in the base body 102 moves the threads 104 (tensile force), continuously and with constant force. The force exerted by the winch is transmitted via the threads 104 to the nets 104, to the edges 112 of the opened tissue structure, so that the edges 112 of the opened tissue structure 114 are at least partially brought together.

A retraction of the edges 112 is thus reduced.

FIG. 3 shows an exemplary representation of a wound care system according to the invention.

The wound care system according to the invention comprises a device 100 according to the invention, as well as a vacuum dressing system.

The embodiment of an apparatus 100 according to the invention shown in FIG. 1 is shown.

Furthermore, a foam film dressing 110 of a vacuum dressing system is shown.

The vacuum dressing system further comprises a vacuum pump (not shown).

In this embodiment, the device 100 is integrated into the foam sheet dressing 110. Alternatively, the device 100 could be applied to the foam sheet dressing 110.

The function of the device 100 can essentially be found in the description of FIG. 1. By means of the clamping element 106, the retraction elements are moved here essentially parallel to the opened tissue structure 114.

The combination of the vacuum dressing system and the device 100 results in a synergistic effect for the wound healing of the patient.

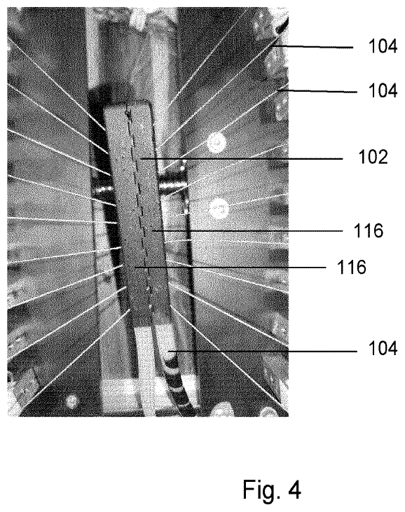

FIG. 4 shows an example of a further embodiment of a device 100 according to the invention for reducing the retraction of the edges and/or for stretching the edges of an opened tissue structure of a patient with an open soft tissue defect (on an abdominal wall simulator).

The abdominal wall simulator here simulates both the opened tissue structure of a patient and the edges of the opened tissue structure.

The device 100 comprises a base body 102.

In this embodiment, the base body 102 comprises two partial bodies 116.

In other words, the base body 102 is divided along its longitudinal axis into two partial bodies 116.

The partial bodies 116 are releasably connected to one another.

The device also includes a plurality of retraction elements 104 for retracting and attaching to the edges of an opened tissue structure.

In this embodiment, the retraction elements 104 comprise threads 104 and tubes 104.

Eighteen threads 104 are shown.

Alternatively and/or additionally, meshes, mesh strips, hooks, clips or similar are conceivable.

Each thread 104 is connected to the abdominal wall simulator, thus simulating a connection of the threads to the edges of the opened tissue structure.

The threads 104 are laterally connected to the abdominal wall simulator, run towards the center of the simulator (corresponding to the center of the patient's body) through a partial body 116 (the nine threads on the left side run through the left partial body 116, the nine threads on the right side run through the right partial body 116, thus allowing the connection of the partial bodies 116 to be separated).

The threads 104 are deflected in the partial bodies 116 and leave the partial bodies 116 on a different side than they entered (here on the underside).

After leaving the partial bodies 116, the threads 104 are encased in tubes 104.

The device also includes a clamping element 106 (not shown).

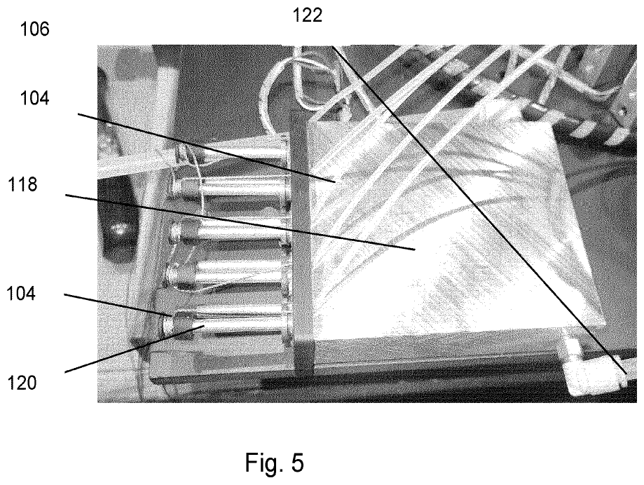

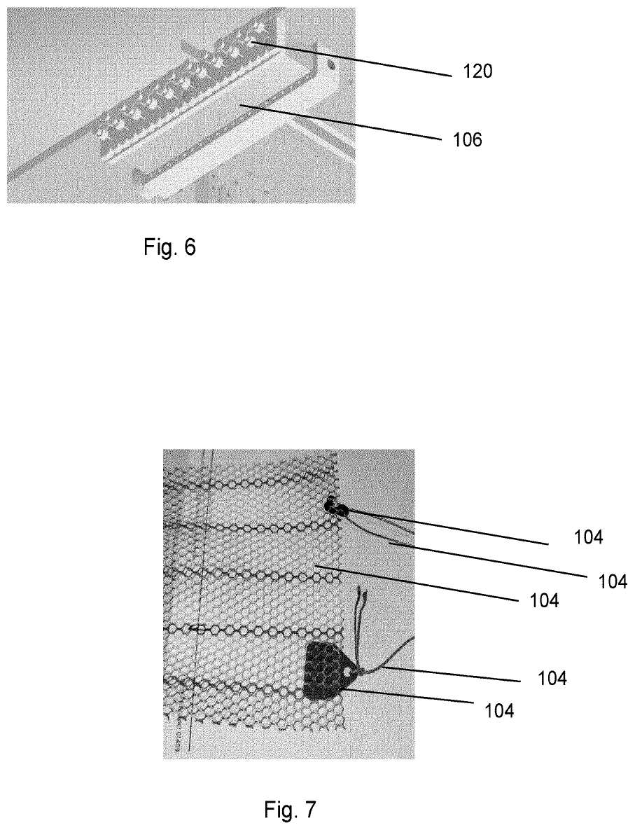

The clamping element 106 includes a chamber 118 (see FIGS. 5 and 6).

In this example, the chamber 118 is a hydraulic chamber 118.

Alternatively and/or additionally, a pneumatic chamber 118 is conceivable.

Eighteen pressure elements 120 are inserted into the chamber 118.

The pressure elements 120 are designed as cylinders 120.

A different number of pressure elements 120 is conceivable, but at least one pressure element 120 is mandatory.

Alternatively, the pressure elements 120 can be connected to the hydraulic chamber 118.

Each pressure element 120 is connected to a thread 104 encased in a tube 104.

In other words, each of the 18 threads is connected at a first end to the abdominal wall simulator and can thus be connected to the edge of an opened tissue structure.

Each of the 18 threads is connected at a second end to a cylinder 120.

A pump is connected to the chamber 118, preferably via a hydraulic and/or pneumatic connection 122.

In this embodiment, the pump is a manual pump.

Alternatively, an electric pump would be conceivable, for example.

The pump can be part of the device 100, but it can also be an external part.

The force transmitted to the cylinders 120 via the chamber 118 by the pump moves the cylinders 120 in such a way that a tensile force is exerted on the threads 104 connected to the cylinders 120 on the abdominal wall simulator to simulate the edges of the opened tissue structure.

The retraction elements 104 (here: threads) can therefore all be moved with the same force via the clamping element 106 (here: chamber 118 and cylinders 120). The force can be predefined and continuous. The presence of a pneumatic chamber means that a patient's coughing jolts can be cushioned by a brief retraction of the cylinders. The force can also be controlled individually and/or as needed using the pump. This allows the opposing parts of the abdominal wall simulator (simulating the edges of the opened tissue structure 114) to be at least partially brought together.

This embodiment comprises 18 threads, but alternatively a different number of threads is conceivable (e.g. 4, 6, 8, 10, 12, 14, 16, 18, 20, 22, 24, 26, and so on). It is advantageous, but not essential, for each partial body to be connected to the same number of threads.

The device 100 described in FIG. 4 can be part of a wound care system 108, which, in addition to the device 100, comprises a vacuum dressing system 108 including a foam-film dressing 110 and a vacuum pump.

A corresponding wound care system can be applied as described below:

A mesh, for example in the form of a mesh strip, is sutured to each of the edges of an open tissue structure of a patient with an open soft tissue defect (e.g. after an operation). It is conceivable, for example, that a mesh is sutured to the posterior rectus sheath, for example a few centimeters lateral to the back of the rectus sheath. A mesh of this type is sutured to the edges of the tissue on two opposite sides of an open tissue structure.

It is advantageous to suture the mesh to the edges of the opened tissue structure with many stitches, so that the force acting on it later can be distributed as evenly as possible over the edges of the opened tissue structure, thus largely preventing it from being torn out of possibly brittle tissue.

A first foam or sponge (part of the vacuum dressing) can be placed in the opened tissue structure. In particular, the first foam or sponge can be placed directly at the outermost edge of the opened tissue structure or under the outermost edge (several centimeters lateral to the outermost tissue edge) to avoid contact of organs, for example intestines, with fascia edges.

Nine clips 104, each connected to a thread 104, are attached to each of the two meshes, preferably on the opposite side of the tissue edge. The nine threads 104 can then be connected to a partial body 116 of the base body 102 of the device 100 as described in FIG. 4.

Nine threads each are deflected in a partial body 116, leave the partial body 116 and are each encased in a tube after leaving it.

The two partial bodies are then detachably connected to one another and placed on the first foam or sponge that was introduced into the opened tissue structure.

A second (subcutaneous) foam or sponge can now be placed on top of this. The tubes with the threads 104 can be routed out at one pole of the opened tissue structure.

A sterile film can now be applied to the second foam or sponge, which ultimately enables the attachment of a suction to the vacuum dressing.

The base body, the first and second foam material and/or sponge are arranged in a sterile manner.

The clamping element 106 can be arranged in a sterile manner under a second film, so that only the pneumatic/hydraulic connection between the clamping element 106 and the pump leaves the sterile space and is connected to the pump.

The pump builds up pressure, which ultimately leads to the pulling of the threads 104, see above.

The vacuum dressing can then be changed as follows:

The connection between the hydraulic chamber 118 and the pump is released.

Next, the second film, the first film and the second foam or sponge are removed.

Care is taken to ensure that the base body 102 remains sterile.

The detachable connection between the two partial bodies 116 is disconnected so that the partial bodies 116, each with their nine threads 104, can be folded to the side without having to detach them from the respective connected mesh strip 104.

The first foam and/or sponge can now be removed and/or replaced. In addition, the opened tissue structure 114 can be examined.

The partial bodies 116 can be reconnected as described above, and the vacuum dressing can be completed as described above.

The base body 116 always remains sterile.

The connection of the clamping element 106 to the pump can be done using a new, sterile connecting tube.

When the therapy is completed, the dressing can be removed as described above.

The clips 104 are removed from the mesh strips 104.

Primary closure of the opened tissue structure can be performed using sutures. The sewn-in mesh strip 104 can be used as a stable suture site in addition.

FIG. 5 shows an example of a clamping element 106 with a hydraulic chamber 118 and five pressure elements 120 in the form of cylinders 120.

The mode of operation of the clamping element 106 is essentially described in FIG. 4, but unlike in FIG. 4, there are only five cylinders 120 here.

A thread 104 is attached to each cylinder 120.

The hydraulic chamber 118 is connected to a pump by a tube 122.

The force transmitted by the pump via the chamber 118 to the cylinders 120 causes the cylinders 120 to move in such a way that a tensile force is exerted on the threads 104 connected to the cylinders 120, which makes it possible to bring the edges of an opened tissue structure closer together.

FIG. 6 schematically shows a further embodiment of a clamping element 106.

In this embodiment, the clamping element 106 comprises a pneumatic chamber 118.

The clamping element 118 further comprises 11 pressure elements.

The functioning of the clamping element 106 is analogous to the clamping elements described in FIGS. 4 and 5.

FIG. 7 shows examples of various retraction elements 104.

Shown are two different hooks 104 fixed to a thread device, which engage in a mesh 104.

A mesh of this kind can be connected (e.g. sewn on) to the tissue edges of an opened tissue structure.

The mesh 104 can be connected to a clamping element 106 via threads 104 attached to the hooks 104.

Two or more interconnected retraction elements 104 can also be considered to be one retraction element 104.

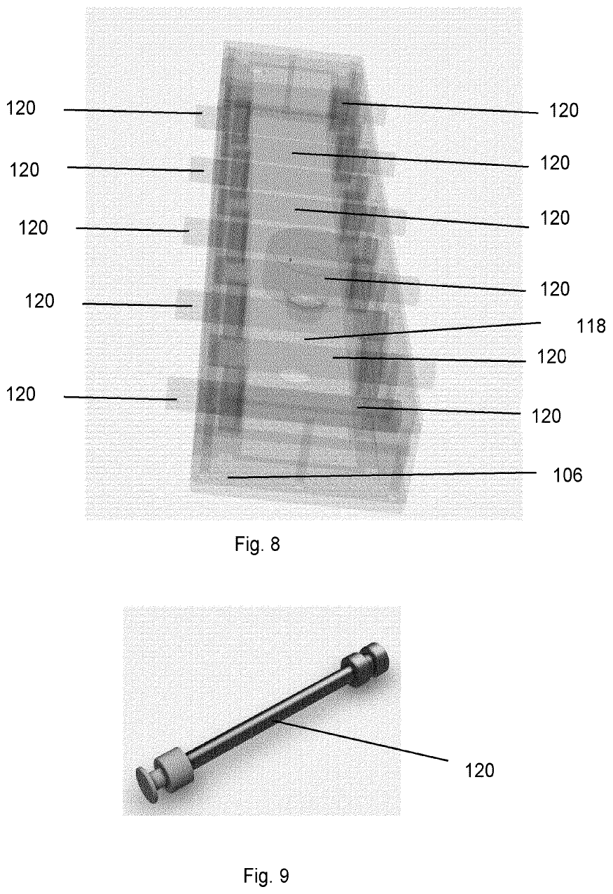

FIG. 8 schematically shows a further embodiment of a clamping element 106.

The clamping element 106 comprises an hydraulic chamber 118 and a total of ten cylinders 120, with five cylinders 120 on two opposite sides of the chamber 118 protruding out of the chamber.

A retraction element 104 can be attached to the head of each cylinder that protrudes from the chamber 118.

Alternatively, the chamber 118 can be a pneumatic chamber 118.

Another number of cylinders 120 is also conceivable.

The mode of operation of the clamping element 106 essentially corresponds to the mode of operation of the clamping element 106 described in FIG. 4.

FIG. 9 shows a detailed view of an exemplary pressure element 120.

A pressure element 120 of this kind can be used in a clamping element 106, as shown for example in FIGS. 5, 6 and 8.

The pressure element 120 is essentially in the form of a cylinder 120.

The head of the pressure element 120 is shown on the left.

This projects out of the chamber 118 (see FIGS. 5, 6) and is designed so that a retraction element 104, in particular in the form of a thread 104, can be easily attached to it.

### Reference Numerals

100 device
102 base body
104 retraction element
106 clamping element
108 wound care system
110 foam-film dressing
112 edge of opened tissue structure
114 tissue structure
116 partial body
118 chamber
120 pressure element/cylinder
122 connection, tube

1. A device for reducing the retraction of the edges and/or for stretching the edges of an opened tissue structure of a patient having an open soft tissue defect, the device comprising at least
one base body,
at least one retraction element for retracting and fastening to the edges of the opened tissue structure
and a clamping element,
wherein the clamping element is designed and arranged in such a way that the at least one retraction element can be moved by means of the clamping element in such a way that the edges of the opened tissue structure are at least partially brought together.

2. A device according to claim 1,
wherein
the device is designed and arranged in such a way that the at least one retraction element can be moved essentially in a plane with the opened tissue structure or essentially parallel to the opened tissue structure by means of the clamping element.

3. A device according to claim 1,
wherein
the clamping element is designed and arranged in such a way that the at least one retraction element can be moved with a force of up to 10 N by means of the clamping element.

4. A device according to claim 1,
wherein
the device comprises at least two retraction elements.

5. A device according to claim 4,
wherein
the base body is arranged essentially centrally between the at least two retraction elements.

6. A device according to claim 1,
wherein
the base body is directly and/or indirectly connected to the at least one retraction element or to the at least two retraction elements at at least one point.

7. A device according to claim 1,
wherein
the base body comprises at least two partial bodies which can be releasably connected to one another.

8. A device according to claim 1,
wherein
a partial body comprises at least one deflecting element that is configured to deflect the at least one retracting element.

9. A device according to claim 1,
wherein
the clamping element comprises a chamber.

10. A device according to claim 1,
wherein
at least one pressure element is connected to the chamber or at least one pressure element is let into the chamber, the pressure element being connected to the at least one retraction element, which is directly or indirectly connected to an edge of the opened tissue structure, wherein the at least one pressure element can be moved by means of a pump connected to the chamber in such a way that a tensile force is exerted on the edge of the opened tissue structure via the at least one retraction element connected to the pressure element.

11. A device according to claim 1,
wherein
the at least one retraction element comprises at least one thread and/or suture material and/or at least one mesh and/or mesh strip and/or at least one clip and/or at least one hook and/or at least one tube and/or at least one wire.

12. A device according to claim 1,
wherein
the device comprises a plurality of retraction elements of at least partially different length and/or size.

13. A wound care system comprising at least one device according to claim 1, wherein the wound care system further comprises a vacuum dressing system, wherein the vacuum dressing system comprises at least one foam sheet dressing and a vacuum pump.

14. Wound care system according to claim 13,
wherein
the at least one device is at least partially integrated into the foam sheet dressing or at least partially applied to it.
